(19) United States
(12) Patent Application Publication
(10) Pub. No.: US 2008/0090222 A1
(43) Pub. Date: Apr. 17, 2008

(54) Virus-Inactivated Hemoglobin And Method Of Producing The Same

(75) Inventors: Tsutomu Ueda, Kanagawa (JP); Tetsuhiro Kimura, Kanagawa (JP); Junya Kojima, Kanagawa (JP)

Correspondence Address: BUCHANAN, INGERSOLL & ROONEY PC, POST OFFICE BOX 1404, ALEXANDRIA, VA, 22313-1404, US

(73) Assignee: Terumo Kabushiki Kaisha, Tokyo (JP)

(21) Appl. No.: 11/631,347

(22) PCT Filed: Jun. 29, 2005
(86) PCT No.: PCT/JP05/11931

§ 371 Date: Dec. 29, 2006

(30) Foreign Application Priority Data
Jun. 30, 2004 (JP) 2004-194884

(51) Int. Cl. A01N 1/00; C07K 14/805

(52) U.S. Cl. 435/2; 530/385

(57) ABSTRACT

[PROBLEMS] To provide a method of efficiently producing virus-inactivated hemoglobin from erythrocytes without affecting physical and chemical properties of hemoglobin while allowing to conduct an erythrocyte hemolysis treatment and viral inactivation treatment at the same time and also to perform a post-hemolysis purification step and a post-virus-inactivation purification step in a single step, and especially, a method of efficiently obtaining highly virus-inactivated and sterile hemoglobin assured of the inactivation of any virus regardless of the presence or absence of envelopes.

[SOLVING MEANS] A method of producing virus-inactivated hemoglobin, which includes: an SD treatment step of bringing erythrocytes and a mixture of a solvent and a detergent into contact with each other to simultaneously conduct an erythrocyte hemolysis treatment and viral inactivation treatment of said erythrocytes and a purification step of collecting virus-inactivated hemoglobin from the resulting SD-treated solution. A final filtration step can be efficiently performed when the purification step is performed in the order of an adsorption treatment and ultrafiltration.

### TECHNICAL FIELD

This invention relates to a method capable of efficiently producing virus-inactivated hemoglobin from erythrocytes, and preferably to a method of producing virus-inactivated hemoglobin, which can efficiently obtain highly virus-inactivated and sterile hemoglobin assured of the inactivation of any virus regardless of the presence or absence of envelopes.

### BACKGROUND ART

Hemoglobin exists in blood, surrounded by surrounded by red blood cell membranes, which are called stromata. Upon processing blood hemoglobin to use it as a blood product or the like, it is therefore necessary to obtain stroma-free hemoglobin (SFH) from collected blood. SFH can be obtained by conducting separation and purification subsequent to hemolysis or disruption of stromata. The hemolysis treatment is, however, limited to conditions that do not denature hemoglobin. It has been the conventional practice to perform a hemolysis treatment by the osmotic pressure method. A hemolysis step, which relies upon the osmotic pressure method, typically includes the following consecutive steps: 1) removing platelets, leukocytes and plasma components from collected natural whole blood to separate and wash only erythrocytes, 2) adding a great deal of distilled water or a hypotonic buffer (for example, phosphate buffer) to disrupt stromata, 3) removing erythrocyte cell-substrata such as stromata and a blood group substance to obtain a high-purity hemoglobin (SFH) solution, and 4) adjusting an electrolyte concentration of the solution to its normal level in the body (see Patent Document 1).

Upon processing hemoglobin, which has been derived from blood as described above, into a blood product or the like and administering it to man for a therapeutic purpose, it is also necessary to assure the sterility and viral inactivity of the product. Especially in view of the AIDS calamity by blood products, the importance of the viral inactivity of a product to be intravenously administered to man is strongly recognized.

There are various methods for the inactivation of viruses, which can be roughly divided primarily into viral inactivation by energy, physical treatments, and chemical treatments. Known viral inactivation by energy include a heating treatment (see Patent Document 2), an ultra-short time heat treatment by microwave irradiation (see Patent Document 3), an ultraviolet ray irradiation treatment (see Patent Document 4), photosentizing effects making use of a photosensitizer such as dimethyl methylene blue (DMMB)(see Patent Document 5), etc. For example, viral inactivation of an albumin product includes a heat treatment at 60° C. for 10 hours. Viral inactivation by energy, however, involves a potential problem of hemoglobin denaturation, so that a limitation is imposed on its application to the treatment of hemoglobin-containing products. For the inactivation of hemoglobin, there is, accordingly, a demand for a method that inactivates viruses but keeps hemoglobin proteins substantially free from denaturation.

A typical example of the physical treatments is size exclusion, and is “nano-filtration (NF)” that filters off a virus by a filter having an extremely small pore size sufficient to remove the virus (called “virus removal membrane”)(see Patent Document 6).

The chemical treatments are known to include a low pH treatment and a chemical treatment making use of a nucleic acid intercalator. A typical example of the chemical treatments is, however, a viral inactivation method which makes use of a biocompatible solvent or detergent, and is also called “the solvent detergent method” (which may hereinafter be also called “the SD treatment method”) (see Patent Document 7 and Non-patent Document 1). The principle of the inactivation of a virus by the SD treatment is to disrupt the shells of an envelope virus with a detergent and to dissolve the former virus in a solvent (see Non-patent Document 1). According to the SD treatment method, the effects of a solvent and a detergent on the lipid envelops of a virus are synergistically promoted owing to the combined use of the solvent and the detergent. The SD method is effective for the inactivation of viruses having lipid envelopes, and is applied to the viral inactivation treatment of blood coagulation factor VIII products.

In the above-described SD treatment method, the used solvent and detergent are generally removed from the treated solution subsequent to the SD treatment. There are several methods for removing the solvent and detergent from the SD-treated solution to levels permissible to a man or certain biological system, and the oil extraction method, the dialysis method and the adsorption method are generally used. For oil extraction, a plant or animal oil or an equivalent synthetic oil is used (see Patent Document 8). The dialysis method is generally the hollow-fiber dialysis method. Known examples of the adsorption method include a method that uses a synthetic adsorbent having no functional group (see Patent Document 9) and chromatography making use of silica beads filled with a three-dimensionally crosslinked, hydrophobic acrylic acid polymer (see Patent Document 10).

The viral inactivation method based on the above-described physical treatment or chemical treatment is applicable to hemoglobin and hemoglobin-containing products. However, the physical treatment and chemical treatment are each accompanied by both merits and demerits and, when applied singly, are difficult to completely remove or completely inactivate various viruses. For example, the above-described SD method is known to be a useful method that can easily and efficiently inactivate viruses having lipid envelopes, such as HIV, HBV and HCV, without needing to heat a product. The SD method is, however, ineffective for the inactivation of viruses having no envelope. With the SD method alone, it is not considered possible to assure the viral inactivation of a hemoglobin product.

Patent Document 1: Japanese Patent Laid-open No. Hei 2-178233

Patent Document 2: Japanese Patent Laid-open No. 2002-112765

Patent Document 3: Japanese Patent No. 2668446

Patent Document 4: Japanese Patent Laid-open No. Hei 11-286453

Patent Document 5: JP-A-2001-514617

Patent Document 6: Japanese Patent Laid-open No. 2002-114799

Patent Document 7: Japanese Patent Laid-open No. Sho 60-51116

Patent Document 8: Japanese Patent No. 2544619

Patent Document 9: Japanese Patent Laid-open No. 2002-34556

Patent Document 10: Japanese Patent Laid-open No. 2001-99835

Non-patent Document 1: Transfusion, 25(6), 516 to 22 (1985 November-December)

### DISCLOSURE OF INVENTION

### Problems to be Solved by the Invention

As described above, the conventional methods of obtaining virus-inactivated hemoglobin from blood each includes independently conducting the individual steps of hemolyzation of erythrocytes separated from blood, purification, and viral inactivation, and each includes many and long overall steps. No proposal has, however, been made yet about a process for the production of virus-inactivated hemoglobin, which can be performed as an efficient continuous process, especially a method of conducting the hemolysis and purification steps with the viral inactivation step in view. In addition, the conventional viral inactivation step is generally performed based on a single treatment method, and an improvement is desired to assure the complete removal or complete inactivation of various viruses. Viral inactivation treatments of different mechanisms have, however, been performed in combination to date with a view to assuring sterile hemoglobin with various viruses inactivated. Specifically, no proposal has been made yet about a combination of a chemical viral-inactivation treatment by the SD treatment method and a physical virus-removing treatment by another method, for example, nano-filtration, to say nothing of a process of efficiently obtaining sterile and virus-inactivated hemoglobin without physical or chemical denaturation of the hemoglobin by the above-described combination.

### Means for Solving the Problems

The present inventors have conducted an investigation about a method of efficiently producing from erythrocytes hemoglobin assured of viral inactivation. In the course of proceeding with an extensive study to establish a method for the production of virus-inactivated hemoglobin, including at least an SD treatment step, an idea occurred that the SD treatment for viral inactivation could be applied directly to stroma-containing erythrocytes. Use of a mixture of a solvent and a detergent is considered possible to dissolve stromata as phospholipid cell membranes. It had, however, been conjectured that, even if inactivation effects on an envelope-containing virus were assured for stroma-free hemoglobin (SFH) in the SD treatment making use of a solvent and detergent at biologically permissible concentrations, no virus-inactivation effects would be expected from a direct application of the SD treatment to erythrocytes in which stromata exist at an overwhelmingly high concentration compared with the virus. Contrary to the expectation, however, viral inactivation was confirmed to be effective when blood was subjected directly to the SD treatment. At the same time, actual effectiveness of the hemolysis treatment in that SD treatment was also confirmed. In other words, it was found that the direct SD treatment of erythrocytes would make it possible to achieve viral inactivation concurrently with hemolysis. It was also confirmed that in the SD treatment, hemoglobin would remain substantially free from denaturation and the methemoglobin reductase system would also remain substantially free from denaturation.

Based on those findings, the present inventors conducted a further extensive investigation toward a process that would apply a physical viral-inactivation treatment to an SD-treated solution to obtain hemoglobin assured of viral inactivation and would permit efficiently conducting all steps including the purification step for the SD-treated solution. As a result, it was found that by conducting two steps out of various purifications steps, that is, adsorption with an adsorbent and ultrafiltration in this order as the purification step after the SD treatment, the subsequent nano-filtration would be successfully conducted with efficiency. That finding has then led to the completion of the present invention as will be described hereinafter.

The present invention provides a method of producing virus-inactivated hemoglobin, which includes: an SD treatment step of bringing erythrocytes and a mixture of a solvent and a detergent into contact with each other to simultaneously conduct an erythrocyte hemolysis treatment and viral inactivation treatment of the erythrocytes, and a purification step of collecting virus-inactivated hemoglobin from the resulting SD-treated solution.

The erythrocytes are generally obtained by centrifugation of whole blood. The SD-treated solution, therefore, contains the solvent, the detergent, and unnecessary substances derived from blood, such as stromata and a blood group substance, in addition to hemoglobin.

In a preferred embodiment, the solvent and detergent used in the foregoing can be tri-(n-butyl) phosphate and a nonionic detergent, respectively.

In the present invention, it is preferred to conduct, as the purification step, an adsorption treatment with an adsorbent and ultrafiltration in this order. The adsorbent can preferably be a synthetic adsorbent.

In another preferred embodiment, the synthetic adsorbent can specifically include a copolymer of styrene and/or an acrylic compound and divinylbenzene.

The ultrafiltration may preferably be conducted using an ultrafiltration membrane of a pore size that has a molecular-weight cutoff of approx. 100,000. In general, the material of this ultrafiltration membrane may be made of a regenerated cellulose and/or a polyethersulfone.

Although a detailed description will be made subsequently about the background of the selection of the above-described combination and order of the two steps as the purification step in the present invention, this purification step is a specific purification step that is important to efficiently conduct sterile filtration as a final step to be generally performed in the method of the present invention, especially to efficiently conduct the present invention to assure a high yield in an embodiment that includes nano-filtration as a preceding step of sterile filtration. In other words, there is a significant production-related difference in that the purification of the SD-treated solution in the present invention requires the separation of the unnecessary substances derived from blood, such as stromata and the blood group substance, together with the solvent and detergent from the hemoglobin while the conventional purification of SFH from the SD-treated solution requires primarily the removal of the solvent and detergent. It is here that the purification step for efficiently conducting the whole process, including the nano-filtration as a subsequent step, has become important. In the purification step according to the present invention, ultrafiltration in which a limitation is imposed on the pore size can be efficiently performed by firstly conducting the adsorptive removal of the solvent, detergent and blood-derived unnecessary substances with an adsorbent especially a synthetic adsorbent, and moreover, these components to be removed, especially the solvent and detergent can be removed to levels permissible to a man or certain biological system in which the bioproduct is used.

In a further preferred embodiment of the method according to the present invention for the production of virus-inactivated hemoglobin, the method additionally includes a filtration step of conducting nano-filtration and sterile filtration in this order subsequent to the above-described purification step.

Viruses are removed by this nano-filtration, and a high level of viral inactivation is achieved by this nano-filtration and the above-described SD treatment. Specifically, the nano-filtration can be performed with a membrane having a pore size of approx. 15 to 70 nm and made of a regenerated cellulose and/or PVDF.

For the sterile filtration, a sterilization filter (sterilization membrane) is used. Specifically, the sterile filtration can be performed with a sterilization filter made of at least one material selected from a regenerated cellulose, a polyethersulfone or PVDF and having a pore size of 0.2 μm.

In the above-described filtration step, ultrafiltration of the nano-filtrate may be conducted as a concentration step before the sterile filtration as needed. For this concentration, it is preferred to conduct ultrafiltration at a pore size having a molecular-weight cutoff of approx. 10,000 to 30,000. By such ultrafiltration, the hemoglobin concentration can be increased to 45 w/w % or higher. From a standpoint of actual handling ease, a hemoglobin concentration of 45 w/w % or so is sufficient. To the resulting concentrate, sterile filtration can be applied.

The present invention also provides highly virus-inactivated hemoglobin, which has been obtained by the production method including the above-described nano-filtration and has been highly virus-inactivated and sterilized. Its concentrate the hemoglobin concentration of which is 45 w/w % or higher is also provided.

Incidentally, the expression “highly virus-inactivated” means that viruses have been inactivated or removed regardless of the presence or absence of envelopes.

### EFFECTS OF THE INVENTION

According to the present invention, the direct SD treatment of erythrocytes can simultaneously conduct both inactivation treatment and hemolysis treatment of viruses without affecting the physical and chemical properties of hemoglobin, and moreover, can achieve the hemolysis of erythrocytes with a smaller amount of the hemolyzing agent (SD mixture) than the conventional hemolysis treatment by the osmotic pressure method. Owing to the direct SD treatment, hemolysis and inactivation which have heretofore been performed independently can be performed as a series of related steps. Described specifically, the purification step as a pre-treatment for hemolysis and the purification step as a post-treatment for viral inactivation can be conducted as a single purification step.

When this single purification step is performed by combining the use of an adsorbent, especially a synthetic adsorbent and an ultrafiltration operation in this order, it is possible not only to efficiently perform this purification step but also to improve the efficiency of a nano-filtration treatment added as a preferred embodiment of the present invention after the purification step, that is, to reduce the treatment time and to improve the yield. Further, this preferred embodiment makes it possible to obtain highly virus-inactivated and sterile hemoglobin assured of the inactivation of any virus regardless of the presence or absence of envelopes.

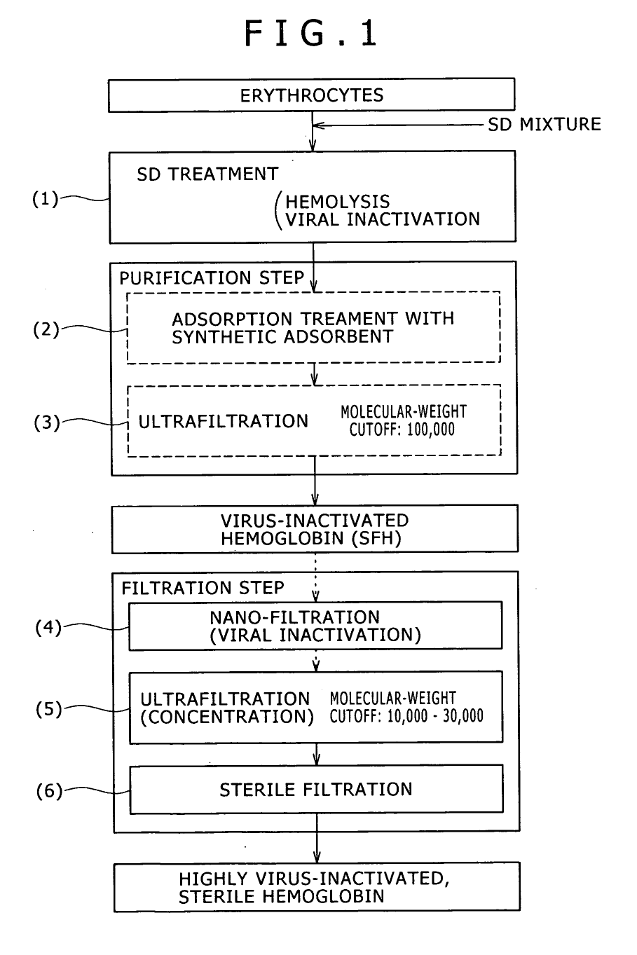

### BRIEF DESCRIPTION OF THE DRAWINGS

[FIG. 1] A diagram schematically illustrating, as a preferred embodiment, a process flow of a method according to the present invention for the production of virus-inactivated hemoglobin.

### BEST MODES FOR CARRYING OUT THE INVENTION

With reference to the process flow illustrated in FIG. 1, the present invention will hereinafter be described specifically. FIG. 1 is a diagram schematically illustrating a process flow by taking, as an example, a particularly preferred embodiment of the present invention, and therefore, the scope of the present invention shall not be limited to the diagram. Nonetheless, the present invention includes at least an SD treatment and a purification step in the diagram. In the diagram, preferred steps and flows are indicated by chain lines.

In the present invention as illustrated in the diagram, upon producing virus-inactivated SFH hemoglobin from erythrocytes having stromata, the SD treatment of step (1) is firstly applied to the erythrocytes.

The SD treatment (1) is a step that brings erythrocytes and a mixture of a solvent and a detergent (hereinafter referred to as “the SD mixture”) with each other. These erythrocytes are generally obtained by centrifugation of whole blood. Specifically, erythrocytes are obtained by separating platelets, leukocytes and plasma components from blood collected from human donors or animals, and are available as an erythrocyte concentrate.

By this contact with the SD treatment solution, the hemolysis treatment and the viral inactivation treatment can be conducted at the same time without causing denaturation of hemoglobin. It is to be noted that no particular limitations are imposed on the SD mixture or contact conditions to be used in this step insofar as the SD mixture can be removed by the synthetic adsorbent treatment and ultrafiltration operation as subsequent steps.

Within a range that satisfies the above-described conditions, the SD mixture can be any solvent-detergent combination known in the technical field of solvent detergents that can generally perform chemical inactivation of viruses having envelopes.

Exemplified specifically, the solvent can be an organic solvent, especially a dialkyl or trialkyl phosphate having C₁₋₁₀ alkyl groups, with a trialkyl phosphate having C₂₋₁₀ alkyl groups being preferred. Specific examples include tri-(n-butyl) phosphate (which may hereinafter be referred to as “TNBP”), tri-(t-butyl) phosphate, tri-(n-hexyl) phosphate, tri-(2-ethylhexyl) phosphate, tri-(n-decyl) phosphate, and ethyl-di(n-butyl) phosphate. In particular, a trialkyl phosphate, for example, tri-(n-butyl) phosphate (which may hereinafter be referred as “TNBP”) can be used preferably.

As the detergent, one capable of dispersing 0.1 wt % of fat in a 0.01 g/mL solution at room temperature can be used generally. Specifically, a polyoxyethylene derivative of a fatty acid, a polyoxyethylene sorbitan fatty acid ester, an oxyethylated alkyl phenol, a polyoxyethylene alcohol, a polyoxyethylene oil, a polyoxyethylene oxypropylene fatty acid, or the like can be mentioned. More specific examples include nonionic detergents such as polyoxyethylene derivatives of fatty acids, e.g., “Tween 80” and “Tween 20” (trade names); partial esters of sorbitol anhydrides, e.g., “Polysorbate 80” (trade name); oxyethylated alkyl phenols, e.g., polyoxyethylene octylphenyl ether (“Triton X-100”, trade name); sulfobetains, e.g., sodium cholate, sodium deoxycholate and N-dodecyl-N,N-dimethyl-2-ammonio-1-ethanesulfonate; and octyl-β,D-glucopyranoside.

In particular, nonionic, oil-soluble, aqueous detergents such as “Tween 80”, “Triton X-100” and sodium cholate can be used preferably.

The SD mixture may respectively contain two or more solvents and/or two or more detergents. The SD mixture may also contain one or more other additives to promote its effects, for example, a reducing agent as needed.

The SD mixture may contain the solvent (S) and detergent (D) in such amounts as giving an S/D (w/w ratio) of 1 to 20.

Although the hemolysis of erythrocytes in their entirety by the SD treatment of the erythrocytes can be fully recognized even by visual observation, it can be confirmed by centrifuging the SD-treated solution of the erythrocytes and analyzing the hemoglobin concentration of the supernatant. The present inventors have also confirmed that the SD treatment does not affect the physical or chemical properties of hemoglobin, hemoglobin proteins remain substantially free from denaturation, and reductase proteins are retained at high level.

The SD treatment of erythrocytes with such an SD mixture can dissolve the lipid envelopes of viruses and can inactivate the viruses owing to synergistic effects of the coexisting solvent and detergent. Accordingly, this SD step is effective for the inactivation of viruses having lipid envelopes, such as HIV, HBV and HCV. In addition, the above-described SD treatment has hemolytic effects for erythrocytes, and is also effective for the disruption of stromata. Moreover, the amount of the SD mixture to be used relative to erythrocytes is smaller compared with that in the conventional hemolysis treatment by the osmotic pressure method, so that the hemolysis of erythrocytes is feasible with a smaller amount of the hemolyzing agent (SD mixture).

In the present invention, it is desired for the assurance of the hemolytic effects and viral inactivation to use the SD mixture in such an amount that the solvent and detergent amount to 0.3 to 1 and 0.05 to 1, respectively, per 100 of the amount of erythrocytes in the contacting system.

The SD treatment can be conducted by bringing a solution of erythrocytes and the SD mixture into contact with each other at a temperature of 0 to 40° C., preferably 4 to 25° C., more preferably 7 to 12° C. The effects of the contact generally appear in several minutes of contact, and the contact may be continued preferably 10 minutes or longer but not longer than two hours, typically for 30 to 60 minutes or so. It is preferred to limit the contact to two hours or shorter, because the SD treatment is not expected to bring about any extra effects even when continued for a time longer than two hours.

As the efficiencies of the hemolysis and viral inactivation by the SD treatment are practically unaffected by the temperature, the SD treatment is set at the above-mentioned temperature from the viewpoint of the stability of hemoglobin, especially the inhibition of conversion into methemoglobin.

In the SD treatment, stromata are disrupted by hemolysis. In such an SD-treated solution, there exist the used solvent and detergent and unnecessary substances derived from the hemolyzed blood, such as stromata and the blood group substance, together with hemoglobin separated from stromata (SFH). It is, therefore, necessary to purify the SD-treated solution to separate and recover the virus-inactivated SFH.

The purification step is a step that separates and recovers the hemoglobin (SFH) from the SD-treated solution. From a specific investigation to be described subsequently herein as Examples, it has been found preferable to conduct, as the purification step, (2) an adsorption treatment with a synthetic adsorbent and (3) ultrafiltration in this order in the present invention.

In a preferred embodiment of the step (2), the synthetic adsorbent can be particles made of a synthetic polymer containing no functional groups, for example, a copolymer of styrene and/or an acrylic compound and divinylbenzene. Usable examples of such a synthetic adsorbent include commercial products available in the names of “DIAION HP Series”, “DIAION SP200 Series”, “DIAION HP1MG” and “DIAION HP2MG” (all, Mitsubishi Chemical Corporation); and “Amberlite XAD (registered trademark) Series” (Rohm & Haas Company). Among these, preferred are “Amberlite XAD (registered trademark) Series” (XAD-16HP, XAD-1180, XAD-2000) which are copolymers of styrene or acrylic compounds and divinylbenzene, respectively.

As the amount of the synthetic adsorbent to be used and the treatment time, a desired concentration and time can be chosen in view of removing effects and economy.

The synthetic adsorbent has strong resistance to alkali and heat, so that a sterilization operation under 121° C. condition by immersion in an aqueous solution of an alkali is feasible and therefore, the product can be assured of pyrogen-free quality and sterility. As the alkali, sodium hydroxide can be used preferably.

By the adsorption treatment (2) with the synthetic adsorbent, large majorities of the added solvent and detergent are removed out of the target substances which are contained in the SD-treated solution and are to be removed. In addition, the removal of the blood-derived stromata and blood group substance is feasible.

The ultrafiltration of the step (3), which is to be conducted next, is also called “cross-flow filtration” or “tangential flow filtration (TFF)”, and different from dead-end filtration which is a method performed in sterile filtration or the like, is a method that a solution is caused to flow in parallel with the surfaces of filter membranes to conduct filtration while removing deposit. Described specifically, filter membrane cassettes useful in cross-flow filtration have a structure that filter membranes are stacked in the form of flat membranes. A solution under treatment flows between and in parallel with the flat membranes. Particles deposited on the surfaces of the flat membranes are washed off by the streams of the solution under treatment, which flows in parallel with the flat membranes. The occurrence of gel layers by the deposition of particles can, therefore, be avoided so as to permit stable filtration.

As the material of the filter membrane or membranes, a regenerated cellulose such as cellulose acetate and/or a synthetic polymer such as a polyethersulfone is generally preferred. As the filter membrane or membranes, one or those having a molecular-weight cutoff and pore size commensurate with the object are used. The pore size should be adequately determined depending upon the sizes of certain viruses and substances not needed to be retained in the solution under treatment, to say nothing of the size of a substance to be retained in the final product and those of the solvent and detergent to be removed. A pore size having a molecular-weight cutoff of about 100,000 is particularly suited for the purpose of removing stromata and the blood group substance to improve the processing efficiency of the nano-filtration as the succeeding step.

In a cross-flow filtration system, it is possible to perform an operation with a membrane area commensurate to the amount of the solution under treatment by controlling the effective filtration area of filter membrane cassettes installed in the filtration system. In other words, it is possible to meet scales of from a small capacity to a large capacity by controlling the number of filter membrane cassettes to be installed one over the other in the filtration system in accordance with the amount of the solution to be treated.

Moreover, the use of a cross-flow filtration system in the present invention permits inline sterilization so that as in the synthetic adsorbent treatment (2), the treated solution can be assured of pyrogen-free quality and sterility. As an inline sterilization method, it is a common practice to recirculate high-temperature steam or an aqueous solution of an alkali, which has been heated to approx. 50° C., through the system. As the alkali, sodium hydroxide can be used preferably.

Cross-flow filtration includes the batch method and the diafiltration method. According to the batch method, filtration is conducted without controlling the amount of a solution of a necessary substance under recirculation through a cross-flow filtration system. At a stage that the amount of the solution has decreased to a predetermined amount, the amount of the solution under recirculation is increased to back with a dispersion medium to another predetermined amount. The above procedure is then repeated. According to the diafiltration method, on the other hand, the amount of a dispersion medium to be supplied corresponding to the amount of the solution to be filtered out is controlled to maintain at a predetermined level the amount of the solution under recirculation. Both of these methods can be used in the present invention. No limitation is imposed on the dispersion medium insofar as it is a solvent capable of stably dispersing and dissolving a necessary substance. Further, no limitation is imposed as to the presence or absence of components such as an osmotic pressure adjuster and pH adjuster in the dispersion medium insofar as they do not have any action of deteriorating or destructing the cross-flow membranes.

The ultrafiltration (3) can remove the solvent and detergent, and hemolyzed-blood-derived, unnecessary substances, which have not been removed by the above-described adsorption treatment (2), to levels permissible to a man or certain biological system in which the hemoglobin is used. By the ultrafiltration, viruses can also be removed to certain extent owing to the size exclusion.

The amounts of the solvent, detergent, stromata and/or blood group substance, which are to be removed by the ultrafiltration (3), can be determined by also taking into consideration conditions for the synthetic adsorbent treatment, such as the amount of the synthetic adsorbent to be used in the preceding step (2) and the treatment time.

By successively conducting (2) the adsorption treatment with the synthetic adsorbent and (3) the ultrafiltration after the SD treatment step (1), the efficiency of the ultrafiltration can be improved, in other words, the treatment time can be reduced and the yield can be improved, thereby making it possible to obtain enveloped-virus-inactivated hemoglobin (SFH). In addition, the efficiency of a filtration treatment step the addition of which after the ultrafiltration is preferred, specifically nano-filtration (4) can be improved, in other words, the treatment time can be reduced and the yield can be improved.

As mentioned above, there are several purification methods for an SD-treated solution. From the standpoint of the process efficiency of production, no particular limitation is imposed on the SD treatment for conventional SFH hemoglobin. In the present invention that erythrocytes are directly subjected to SD treatment, however, it has been found as a result of a detailed investigation by the present inventors that from the viewpoint of production efficiency, it is a particularly preferred embodiment to conduct the above-described step (2) and step (3) in combination in this order.

It is to be noted that with only the adsorption step (2) by the synthetic adsorbent, the added solvent and detergent can be hardly removed substantially in their entirety or can be hardly removed substantially to levels permissible to a man or certain biological system in which the bioproduct is used, further to such levels as substantially preventing the clogging of the micropores of a nano-filter in the nano-filtration. There are also problems in that the synthetic adsorbent is required in a large amount for the adsorption treatment by chromatography and the adsorption-treated product can be hardly assured of sterility.

Direct ultrafiltration (3) of the SD-treated solution as is, on the other hand, causes the clogging of the filters with the solvent, detergent, stromata and the like, and as a result, leads to an increase in filtration time and an increase in the membrane area of the costly filters, and requires highly frequent filter replacements, leading to a further reduction in yield.

As a further purification method, there is the oil extraction method. According to the oil extraction method, a mixture occurred as a result of the addition of an oil is stirred. By settling or centrifugation, the mixture is then caused to separate into an upper layer and lower layer, and the upper layer is decanted off. If it is desired to perform nano-filtration after the SD treatment, there is a need to sufficiently remove the added oil together with the solvent, detergent and hemolyzed-blood-derived, unnecessary substances prior to the nano-filtration. The existence of the oil component, even in at a trace amount, clogs the micropores of the nano-filter, and as a result, increases the filtration time, requires highly frequent replacements of the costly filter, and has a general tendency to reduce the yield of the product. Further, a great deal of oil may be required depending on the efficiency of extraction. Concerning the detergent, however, its efficient removal is difficult intrinsically.

In the hollow-fiber dialysis method, a solvent clogs the small pores of hollow fiber, and reduces the efficiency of dialysis. A detergent, on the other hand, forms a polymeric micelle, and therefore, makes the efficiency of dialysis very poor and requires a long time and a great deal of an outer dialyzate. The dialysis method is basically a method, which is useful for accuracy control substances or standard substances to be employed in clinical tests or which is useful in their production processes or as a method for improving clinical test, is useful in preparation methods of samples. The dialysis method is, therefore, considered to be unsuited for bulk processing in which a target substance is diluted by dialysis.

In the present invention, the virus-inactivated hemoglobin (SFH) obtained as described above is generally subjected to sterile filtration (6) as a final step to provide a product. It is, however, desired to conduct a physical virus-removing treatment by performing the nano-filtration (4) preferably before the final sterile filtration (6).

The filter for use in the nano-filtration (4) can be a hollow-fiber microporous membrane generally made of a regenerated cellulose, a PVDF filter, or the like.

The removal of viruses by the nano-filtration is achieved primarily by the mechanism based on multi-sieve effects, in other words, by the physical removal of virus particles. The pore size should, therefore, be appropriately determined depending upon the size of a substance to be retained in the final product and the sizes of viruses to be removed by size exclusion. In embodiments of the present invention, “Ultipor VF DV20” (product of Pall Corporation) or “Viresolve NFP” (product of Millipore Corporation) can be effectively used.

As one of critical parameters for nano-filtration, the amount of impurities contained in the final product can be mentioned. In the present invention, the solvent and detergent added at the beginning and unnecessary substances such as the hemolyzed-blood-derived stromata and blood group substance are removed by the combination of the use of the synthetic adsorbent and the ultrafiltration operation as a preceding purification step. The present invention has, therefore, made it possible to improve the efficiency of the nano-filtration treatment for the provision of the product highly purified to meet the critical parameters, specifically to reduce the treatment time and to improve the yield.

In the present invention, the treated solution to be subjected to the sterile filtration (6), preferably the nano-filtrate can be subjected to the ultrafiltration (5) to concentrate the same as needed.

This ultrafiltration (5) can be conducted basically by using a similar system as in the ultrafiltration (3) in the above-described purification step, that is, cross-flow filtration or tangential flow filtration (TFF). As filter membranes for use in the ultrafiltration, those having a pore size commensurate to the degree of concentration are used. At the same time, their molecular-weight cutoff and pore size should be appropriately determined depending upon the size of a substance to be retained in a final product and the sizes of substances to be removed. When concentrating hemoglobin, a pore size having a molecular-weight cutoff of approx. 10,000 to 30,000 is suited.

In the present invention, concentration to a hemoglobin concentration of 45 w/w % or higher is feasible by this ultrafiltration step (5).

In the present invention, sterile filtration (6) by a sterilization filter having a pore size of 0.2 μm is conducted for sterilization as a final step. The membrane material of the sterilization filter can be a regenerated cellulose such as cellulose acetate, a polyethersulfone, PVDF, or the like.

This sterile filtration step (6) is a common step required to obtain a sterile hemoglobin product. In the present invention, filtration is still feasible with good filtration characteristics even when a high-viscosity concentrate concentrated to a hemoglobin concentration of 45 w/w % is fed to the sterile filtration step (6).

### EXAMPLES

The present invention will next be specifically described based on Examples. It is, however, to be noted that the following Examples are intended to illustrate the present invention and the present invention shall not be limited to them.

The hemolysis of erythrocytes in their entirety by SD treatment of erythrocytes can be fully recognized even by visual observation. In the following Examples, however, it was confirmed by centrifuging the SD-treated solution of erythrocytes (conditions: 18,000 G×30 minutes) and analyzing the hemoglobin concentration of the supernatant.

It was also confirmed by a biochemical analysis that hemoglobin and a methemoglobin reductase system were neither physically nor chemically denatured by the SD treatment.

### Example 1

### Virus Spike Test

The following Example was conducted to investigate the viral inactivation effects by the SD treatment of erythrocytes and the presence or absence of denaturation of hemoglobin.

<Preparation of SD Mixtures>

SD mixtures of concentrations ten times higher than their corresponding concentrations at the time of a virus spike test were prepared as will be described hereinafter. Firstly, 25 mL aliquots of “Meylon-84” (Otsuka Pharmaceutical Co., Ltd.) were each diluted to 100 mL with injection-grade distilled water to prepare 25 mM solutions of sodium bicarbonate.

A detergent [polyoxyethylene(10) octylphenyl ether (“Triton X-100”; ICN Biomedicals Inc.) or sodium deoxycholate (Wako Pure Chemical Industries, Ltd.)] and tri-(n-butyl) phosphate (TNBP; Wako Pure Chemical Industries, Ltd.) were mixed in predetermined amounts, respectively, to prepare 25 mM solutions of sodium bicarbonate and aqueous solutions in injection-grade distilled water such that the solutions contained them at concentrations ten times higher than their corresponding concentrations at the time of SD treatment as shown in Table 1.

<Preparation of Samples for the Spike Test>

The following two samples were prepared for the spike test.

To an erythrocyte concentrate obtained by removing platelets, leukocytes and plasma components from natural whole human blood, physiological saline was added as much as needed. The resulting mixture was stirred, and subsequent to centrifugation, the lower layer was collected to obtain rinsed erythrocytes. A 20 mM solution (240 g) of sodium bicarbonate was added to the rinsed erythrocytes (65.23 g, 60 mL) to effect hemolysis. The resultant mixture was then centrifuged (10,000 rpm×30 minutes) to obtain a stroma-containing hemoglobin solution. Subsequently, a half of the stroma-containing hemoglobin solution was filtered through a 0.45-μm syringe filter to obtain a stroma-free hemoglobin solution.

<Virus Spike Test>

To aliquots (0.9 mL) of the above-obtained, stroma-containing and stroma-free samples for the test, aliquots (0.1 mL) of a virus (Human herpes virus 1) solution were added, respectively. Subsequent to thorough mixing, the SD mixtures (0.1 mL, each) prepared as described above were added to give a total volume of 1.1 mL, respectively, so that test solutions of the predetermined final concentrations for SD treatment were prepared. Immediately after the preparation, the test solutions were separately mixed at 7 to 10° C. for the corresponding times shown in Table 1, respectively, to perform a viral inactivation treatment. Subsequent to the treatment, the test solutions were cryopreserved (−80° C.) and were provided for the measurement of virus titers.

<Measurement Method of Virus Titers>

For the measurement of each virus titer (TCID₅₀), the Reed-Munch method was used.

RF (virus reduction factor; virus clearance factor) is a value determined by subtracting a common logarithm of a virus titer of a corresponding sample, which had been treated with the solvent and the corresponding detergent (S/D), from a common logarithm of a virus titer of the corresponding untreated sample.

Incidentally, the employed virus, Human herpes virus 1, is medium in physical and chemical resistance properties.

The results are shown in Table 1.

[Table 1]

[Table 2]

TABLE 1

| | | | | Virus titer | | |
|---|---|---|---|---|---|---|
| Conditions for SD treatment | | | | Log₁₀TCID₅₀/1-mL test | | |
| | | | Treatment | solution | | |
| | Detergent | | time | | Test | |
| Solvent | Kind | Concentration | hr | Control | solution | RF |
| TNBP | Triton X-100 | 1% | 12 | 8.0 | ≦3.0 | ≧5.0 |
| 0.3% | | 0.2% | | 8.0 | ≦3.0 | ≧5.0 |
| | | | | 7.2 | ≦3.0 | ≧4.2* |
| | Sodium | 0.2% | | 8.0 | ≦3.0 | ≧5.0 |
| | deoxycholate | 0.05% | | 8.0 | ≦3.0 | ≧5.0 |
| | | | | 7.2 | ≦3.0 | ≧4.2* |
| TNBP | Triton X-100 | 0.05% | 0 | 9.1 | 4.5 | 4.6 |
| 0.3% | | | 0.5 | | 5.5 | 3.6 |
| | | | 1 | | ≦2.8 | ≧6.3 |
| | | | 2 | | ≦2.8 | ≧6.3 |
| | | | 4 | | ≦2.8 | ≧6.3 |
| | | 0.1% | 0 | | 4.1 | 5.0 |
| | | | 0.5 | | 4.1 | 5.0 |
| | | | 1 | | ≦2.8 | ≧6.3 |
| | | | 2 | | ≦2.8 | ≧6.3 |
| | | | 4 | | ≦2.8 | ≧6.3 |
| | | 0.2% | 0 | | 4.5 | ≧6.3 |
| | | | 0.5 | | 5.5 | ≧6.3 |
| | | | 1 | | ≦2.8 | ≧6.3 |
| | | | 2 | | ≦2.8 | ≧6.3 |
| | | | 4 | | ≦2.8 | ≧6.3 |
| | | 0.4% | 0 | | 3.1 | 6.0 |
| | | | 0.5 | | ≦2.8 | ≧6.3 |
| | | | 1 | | ≦2.8 | ≧6.3 |
| | | | 2 | | ≦2.8 | ≧6.3 |
| | | | 4 | | ≦2.8 | ≧6.3 |

*Stroma-free hemoglobin solution

<Assessment>

In the above-described test, the presence or absence of stromata did not affect the viral inactivation effects. When the virus titer of the positive control in the measurement system was 8.0 in the spike test making use of Human hyper virus 1, a virus titer≦3.0 (Log₁₀TCID₁₀/1-mL test solution) and a virus clearance factor (reduction factor)≧5.0 were indicated under the corresponding SD conditions (“Triton X-100”: 0.2%/TNBP: 0.3% mixed solution, 8.5° C./12 hours) regardless of the presence or absence of stromata.

The used detergents each gave an RF of 5.0 or greater. The used detergents each gave good results even on the side of a low concentration upon SD treatment, specifically at 0.2% in the case of “Triton X-100” or 0.05% in the case of sodium deoxycholate.

### Example 2

The following Example 2 was conducted to establish a preferred purification step in the present invention.

<SD Treatment of Erythrocytes>

To an erythrocyte concentrate obtained by removing platelets, leukocytes and plasma components from natural whole human blood, physiological saline was added as much as needed. The resulting mixture was stirred, and subsequent to centrifugation, the lower layer was collected to obtain rinsed erythrocytes.

Sample 1: To the resulting rinsed erythrocytes (200 g), an SD mixture prepared in advance (200 g; an aqueous solution containing 0.6% of TNBP, 2.0% of “Triton X-100” and an adequate amount of sodium bicarbonate) was added. Under conditions that the solvent and detergent concentrations in the whole solution under treatment were 0.3% of TNBP and 1.0% of “Triton X-100”, an SD treatment was conducted (under stirring at 4 to 10° C. for two hours or longer) to obtain a virus-inactivated, hemolyzed sample 1 (400 g).

Sample 2: A virus-inactivated, hemolyzed sample 2 was obtained in a similar manner as in the sample 1 except that the solvent and detergent concentrations in the whole solution under treatment were changed to 0.3% of TNBP and 0.2% of “Triton X-100”.

<Purification>

Synthetic adsorbent treatment: Aliquots (200 g) of the virus-inactivated, hemolyzed samples obtained as described above were each caused to recirculate at a flow rate of 3.2 L/min for two hours through a column packed with “Amberlite (trademark) XAD-16HP” (40 mL; Rohm & Haas Company).

Oil extraction treatment: Separately from the above-described treatment, aliquots (50 g and 30 g) of soybean oil were added to aliquots (50 g and 70 g) of each of the above-described virus-inactivated, hemolyzed samples, respectively, to prepare mixed solutions containing 50% and 30% of the soybean oil, respectively. Subsequently, the mixed solutions were separately subjected to centrifugation (2.63 kG, 12 min, 4° C.), and the lower layers were recovered.

With respect to each sample treated as described above, the residual amounts of the solvent (TNBP) and detergent (“Triton X-100N”) were quantitatively analyzed by gas chromatography in the case of TNBP and by high-performance liquid chromatography in the case of “Triton X-100N”. The results are shown in Table 2.

[Table 3]

TABLE 2

| | | Amounts in tested | | Percent residue in | |
|---|---|---|---|---|---|
| | | material (μg/g) | | tested material (%) | |
| Sample | Treatment | TNBP | Triton X-100N | TNBP | Triton X-100N |
| 1 | SD hemolysis treatment | 3233 | 11574 | 100 | 100 |
| | Synthetic adsorbent treatment | 15.86 | 104 | 0.49 | 0.9 |
| | Oil extraction treatment (50%) | 14.48 | 814 | 0.45 | 7 |
| | Oil extraction treatment (30%) | 28 | 1308 | 0.87 | 11.3 |
| 2 | SD hemolysis treatment | 3090 | 2247 | 100 | 100 |
| | Synthetic adsorbent treatment | 2.9 | 3.8 | 0.09 | 0.17 |
| | Oil extraction treatment (50%) | 9.08 | 126 | 0.29 | 5.61 |
| | Oil extraction treatment (30%) | 18.37 | 194 | 0.59 | 8.65 |

In the table, TNBP: tri-(n-butyl) phosphate

As shown in Table 2, the adsorption treatment with the synthetic adsorbent has been found to have higher effects for the removal of the solvent and detergent than the oil extraction treatment. Concerning the detergent, in particular, the adsorption treatment with the synthetic adsorbent showed very high removing effects than the oil extraction treatment.

### Example 3

Based on the results of Example 2, ultrafiltration was conducted after the adsorption treatment with the synthetic adsorbent to investigate effects for the removal of the solvent, detergent and stromata.

(1) SD Treatment of Erythrocytes

A virus-inactivated, hemolyzed sample (TNBP: 0.3%, “Triton X-100”: 0.2%; 2.0 kg) subjected to an SD treatment in a similar manner as the sample 2 of Example 2 was obtained.

<Purification>

(2) Synthetic adsorbent treatment: The above-described sample was caused to recirculate in its entirety (2.0 kg) at a flow rate of 3.2 mL/min for two hours through a column packed with 500 mL of the synthetic adsorbent, “Amberlite XAD-16HP”.

(3) Ultrafiltration: Using a cross-flow filtration system

(“SARTOCON SLICE FILTER CASSETTE”, manufactured by Sartorius AG) equipped with filter membranes (material: polyethersulfone, pore size: 100,000 in terms of molecular-weight cutoff, effective filtration area: 0.3 m², product of Sartorius AG), ultrafiltration was then conducted under the conditions of a recirculation solution inlet-side pressure of 0.1 MPa, a recirculation solution outlet-side pressure of 0.025 MPa and a transmembrane-solution side pressure of 0 MPa to obtain a transmembrane solution (1.6 kg).

With respect to the resultant transmembrane solution, the residual amounts of the TNBP and “Triton X-100” were quantitatively analyzed in a similar manner as in Example 2. Further, the residual amounts of phosphatidylserine, phosphatidylcholine and sphingomyelin were also quantitatively analyzed as an analysis of stromata by high-performance liquid chromatography. The analysis results are shown in Table 3.

[Table 4]

TABLE 3

| | Residual amounts in tested material (μg/g) | | | | |
|---|---|---|---|---|---|
| | | Triton | | | |
| Purification step | TNBP | X-100 | Phosphatidylserine | Phosphatidycholine | Sphingomyelin |
| (2) Synthetic adsorbent | 3.79 | 60 | 736.9 | 521.2 | 66.0 |
| treatment | | | | | |
| (3) Ultrafiltration | 0.19 | N.D. | N.D. | N.D. | N.D. |

N.D.: Below detection limit

As shown in Table 3, the solvent TNBP was removed to 3.79 μg/g by the synthetic adsorbent treatment (2) or to 0.19 μg/g by the ultrafiltration (3). On the other hand, the detergent “Triton X-100” was removed to 6.0 μg/g by the synthetic adsorbent treatment (2) or to below the detection limit (N.D.) by the ultrafiltration (3).

As an analysis of stromata, phosphatidylserine, phosphatidylcholine and sphingomyelin were each removed to below the detection limit (N.D.) by conducting the synthetic adsorbent treatment (2) and ultrafiltration (3).

By conducting the synthetic adsorbent treatment (2) before the ultrafiltration (3) as described above, stable treatments were feasible from the viewpoints of the time required for the ultrafiltration (3) and the yield.

### Comparative Example 1)

The conventional method that erythrocytes hemolyzed by the osmotic pressure method are subjected to an SD treatment to inactivate viruses was performed to investigate the solvent-removing effects by ultrafiltration.

<Hemolysis>

Rinsed erythrocytes (10 kg), which had been prepared by a similar operation as in Example 2, were added into a mM solution (50 L) of sodium bicarbonate to hemolyze the erythrocytes. After the hemolysis, ultrafiltration was conducted (recirculation solution inlet-side pressure: 0.1 MPa, recirculation solution outlet-side pressure: 0.02 MPa, transmembrane-solution side pressure: 0.01 MPa) by using a cross-flow filtration system (“SARTOCON 2 PLUS”, manufactured by Sartorius AG) equipped with filter membranes (material: “HYDROSART”, pore size: 0.45 μm, effective filtration area: 1.2 m², product of Sartorius AG). To further improve the recovery percentage, water-added filtration was repeated with a 20 mM solution of sodium bicarbonate to obtain a transmembrane solution (90 L).

<SD Treatment>

To the transmembrane solution obtained as described above, an SD mixture (an aqueous solution containing 3.0% of TNBP, 2.0% of sodium deoxycholate and an appropriate amount of sodium bicarbonate) which had been prepared beforehand was added such that the concentrations of the solvent and detergent in the whole treated solution became 0.3% of TNBP and 0.2% of sodium deoxycholate, respectively. After ultrafiltration was conducted again in a similar manner as described above, the resultant transmembrane solution was subjected to ultrafiltration (recirculation solution inlet-side pressure: 0.1 MPa, recirculation solution outlet-side pressure: 0.04 MPa, transmembrane-solution side pressure: 0.01 MPa) by using a cross-flow filtration system (“SARTOCON 2 PLUS”, manufactured by Sartorius AG) equipped with filter membranes (material: polyethersulfone, pore size: 100,000 in terms of molecular-weight cutoff, effective filtration area: 1.4 m², product of Sartorius AG). To further improve the recovery percentage, water-added filtration was repeated with a 20 mM solution of sodium bicarbonate to obtain a transmembrane solution (118 L).

With respect to the resulting transmembrane solution, the residual amount of the solvent TNBP was quantitatively analyzed. Results are shown in Table 4.

[Table 5]

TABLE 4

Residual amount of solvent in
tested material

| | Residual amount | Percent residue |
|---|---|---|
| | of TNBP (μg/g) | of TNBP (%) |
| SD treatment | 3734.54 | 100 |
| Ultrafiltration | 645.05 | 17.3 |
| (0.45 μm) | | |
| Ultrafiltration | 402.96 | 10.8 |
| (100,000) | | |

As shown in Table 4, the percent residue of TNBP was approx. 17.3% after the ultrafiltration at the pore size of 0.45 μm or approx. 10.8% after the ultrafiltration at the pore size of 100,000 in terms of molecular-weight cutoff.

While the recovery percentage of hemoglobin by the ultrafiltration at the pore size of 0.45 μm after the viral inactivation treatment was approx. 85%, the recovery percentage of hemoglobin by the ultrafiltration at the pore size of 100,000 in terms of molecular-weight cutoff was approx. 45%, thereby indicating a significant reduction in recovery percentage.

### Example 4

### Purification of Hemoglobin from Erythrocytes

(1) SD Treatment of Erythrocytes

A virus-inactivated, hemolyzed sample (TNBP: 0.3%, “Triton X-100”: 0.2%; 50 kg) subjected to an SD treatment in a similar manner as the sample 2 of Example 2 was obtained.

<Purification>

(2) Synthetic adsorbent treatment: Into a tank containing the above-described sample (50 kg), the synthetic adsorbent, “Amberlite XAD-16HP”, (12 L) was added. Using “CLEARMIX STIRRER” (manufactured by M Technique, K.K.), the resultant mixture was stirred for 24 hours under the conditions of a rotational speed of 300 Hz and approx. 7 to 10° C.

(3) Ultrafiltration: Using a cross-flow filtration system (“SARTOCON 2 PLUS”, manufactured by Sartorius AG) equipped with filter membranes (material: polyethersulfone, pore size: 100,000 in terms of molecular-weight cutoff, effective filtration area: 4.2 m², product of Sartorius AG), ultrafiltration was then conducted (recirculation solution inlet-side pressure: 0.09 MPa, recirculation solution outlet-side pressure: 0.04 MPa, transmembrane-solution side pressure: 0.02 MPa). To further improve the recovery percentage, water-added filtration was repeated with a 20 mM solution of sodium bicarbonate to obtain a transmembrane solution (147 L).

(4) Nano-Filtration

The thus-obtained transmembrane solution was nano-filtered under a condition of 0.15 MPa by using a virus-removing filter, “Viresolve NFP Opticap Capsule” (product of Millipore Corporation.).

(5) Concentration

By a cross-flow filtration system (“SARTOCON 2 PLUS”, manufactured by Sartorius AG) equipped with filter membranes (material: polyethersulfone, pore size: 30,000 in terms of molecular-weight cutoff, effective filtration area: 1.2 m², product of Sartorius AG), the nano-filtrate was subjected in its entirety to ultrafiltration to obtain a concentrate (approx. 7.5 kg) having a hemoglobin concentration of 45 w/w %.

(6) Sterile Filtration

By a sterilization filter having a pore size of 0.2 μm and intended for sterilization, “SARTOPORE 2” (material: polyethersulfone, effective filtration area: 0.45 m²), the concentrate was subjected to sterile filtration to obtain hemoglobin assured of viral inactivation and sterility.

The residual amounts of the TNBP and “Triton X-100” were quantitatively analyzed in a similar manner as in Example 2. Further, the residual amounts of phosphatidylserine, phosphatidylcholine and sphingomyelin were also quantitatively analyzed as an analysis of stromata by high-performance liquid chromatography.

The residual amounts of the solvent (TNBP) and detergent (“Triton X-100”) after the steps (1) to (4) and as an analysis of stromata after the steps (2) to (4), phosphatidylserine were quantitatively analyzed. The results are shown in Table 5.

[Table 6]

TABLE 5

| | | | Percent | | |
|---|---|---|---|---|---|
| | Amounts in | | residue in | | Stromata in tested |
| | tested material | | tested | | material |
| | (μg/g) | | material (%) | | Residual amount of |
| | | Triton | | Triton | phosphatidylserine |
| | TNBP | X-100 | TNBP | X-100 | (μg/g) |
| (1) SD hemolysis treatment | 2605 | 1958 | 100 | 100 | — |
| (2) Synthetic adsorbent treatment | 6.88 | 5.4 | 0.26 | 0.28 | 352.9 |
| (3) Ultrafiltration | 1.86 | N.D. | 0.07 | N.D. | 0.14 |
| (4) Nano-filtration | 1.65 | N.D. | 0.06 | N.D. | 0.13 |

N.D.: Below detection limit

As show in Table 5, the solvent TNBP was removed to approx. 0.26% by the synthetic adsorbent treatment (2) and to approx. 0.07% by the ultrafiltration (3). On the other hand, the detergent “Triton X-100” was removed to approx. 0.28% by the synthetic adsorbent treatment (2) and to below the detection limit by the ultrafiltration (3). By conducting the synthetic adsorbent treatment (2) and ultrafiltration (3), phosphatidylserine was removed to 0.14 μg/g as an analysis of stromata.

By performing an operation in the order of the synthetic adsorbent treatment (2), the ultrafiltration (3) and the nano-filtration (4) as described above, stable treatments were feasible from the viewpoints of the times required for the ultrafiltration (3) and nano-filtration (4) and the yield.

1. A method of producing virus-inactivated hemoglobin, which comprises:
an SD treatment step of bringing erythrocytes and a mixture of a solvent and a detergent into contact with each other to simultaneously conduct an erythrocyte hemolysis treatment and viral inactivation treatment of said erythrocytes, and
a purification step of collecting virus-inactivated hemoglobin from the resulting SD-treated solution.

2. The method of producing virus-inactivated hemoglobin according to claim 1, wherein said SD-treated solution comprises said solvent, said detergent, erythrocyte-derived stroma and a blood group substance together with said hemoglobin.

3. The method of producing virus-inactivated hemoglobin according to claim 2, wherein as said purification step, an adsorption treatment with an adsorbent and ultrafiltration are conducted in this order.

4. The method of producing virus-inactivated hemoglobin according to claim 2, further comprising, subsequent to said purification step, a filtration step in which nano-filtration and sterile filtration are conducted in this order.

5. The method of producing virus-inactivated hemoglobin according to claim 2, wherein said solvent is tri-(n-butyl) phosphate, and said detergent is a nonionic detergent.

6. The method of producing virus-inactivated hemoglobin according to claim 3, wherein said adsorbent is a synthetic adsorbent made of a copolymer of styrene and/or an acrylic compound and divinylbenzene.

7. The method of producing virus-inactivated hemoglobin according to claim 3, wherein said ultrafiltration is performed using an ultrafiltration membrane made of a regenerated cellulose and/or a polyethersulfone.

8. The method of producing virus-inactivated hemoglobin according to claim 4, wherein said nano-filtration is performed using a membrane having a pore size of approx. 15 to 70 nm and made of a regenerated cellulose and/or PVDF.

9. The method of producing virus-inactivated hemoglobin according to claim 4, wherein said sterile filtration is performed using a sterilization membrane made of at least one material selected from a regenerated cellulose, a polyethersulfone or PVDF and having a pore size of 0.2 μm.

10. The method of producing virus-inactivated hemoglobin according to claim 4, wherein prior to said sterile filtration, a nano-filtrate is ultrafiltered to concentrate the same.

11. Highly virus-inactivated hemoglobin obtained by a production method according to claim 4, wherein viruses have been highly inactivated to sterilize said hemoglobin.

12. Highly virus-inactivated hemoglobin obtained by a production method according to claim 10 and has a hemoglobin concentration of at least 45 w/w %, and wherein viruses have been highly inactivated to sterilize said hemoglobin.

13. The method of producing virus-inactivated hemoglobin according to claim 1, wherein as said purification step, an adsorption treatment with an adsorbent and ultrafiltration are conducted in this order.

14. The method of producing virus-inactivated hemoglobin according to claim 1, further comprising, subsequent to said purification step, a filtration step in which nano-filtration and sterile filtration are conducted in this order.

15. The method of producing virus-inactivated hemoglobin according to claim 1, wherein said solvent is tri-(n-butyl) phosphate, and said detergent is a nonionic detergent.

16. The method of producing virus-inactivated hemoglobin according to claim 3, wherein said solvent is tri-(n-butyl) phosphate, and said detergent is a nonionic detergent.

17. The method of producing virus-inactivated hemoglobin according to claim 16, wherein said adsorbent is a synthetic adsorbent made of a copolymer of styrene and/or an acrylic compound and divinylbenzene.

18. The method of producing virus-inactivated hemoglobin according to claim 16, wherein said ultrafiltration is performed using an ultrafiltration membrane made of a regenerated cellulose and/or a polyethersulfone.

19. The method of producing virus-inactivated hemoglobin according to claim 4, wherein said solvent is tri-(n-butyl) phosphate, and said detergent is a nonionic detergent.
